(19) United States
(12) Patent Application Publication
(10) Pub. No.: US 2018/0221021 A1
(43) Pub. Date: Aug. 9, 2018

(54) BRAIDED SUTURE WITH FILAMENT CONTAINING A MEDICANT

(71) Applicant: Ethicon, Inc., Somerville, NJ (US)

(72) Inventors: Howard Scalzo, Kenilworth, NJ (US); Leo B. Kriksunov, Ithaca, NJ (US); Robert J. Tannhauser, Bridgewater, NJ (US); Emil Richard Skula, Wayne, NJ (US)

(21) Appl. No.: 15/427,963

(22) Filed: Feb. 8, 2017

(51) Int. Cl. A61B 17/06; A61L 17/10; A61L 17/00; A61K 31/085; A61K 31/155; A61K 38/44; A61L 17/04

(52) U.S. Cl. CPC A61B 17/06166; A61L 17/105; A61L 17/005; A61K 31/085; A61B 2017/00526; A61K 38/443; C12Y 101/03004; A61L 17/10; A61L 17/04; A61K 31/155

(57) ABSTRACT

An implantable medical device and method of making it, including a collection of filaments, including a plurality of first variety filaments made of a first polymeric material and at least one second variety filament, and optionally at least one third variety of filament, wherein the second variety filament is coated or impregnated with a first biomedically useful agent and, if present, the third variety of filament is coated or impregnated with a second biomedically useful agent different from the first biomedically useful agent.

### FIELD

The field of art to which this invention relates is medical devices, such as braided multifilament sutures or woven or knitted meshes, more specifically braided surgical sutures made from filaments of multiple polymers having novel properties, including in vivo properties.

### ENVIRONMENT

Surgical sutures and attached surgical needles are well known in the art for use in a variety of conventional surgical procedures. For example, such sutures may be used to approximate tissue about incisions or lacerations in epidermal layers and underlying fascia layers, join blood vessel ends, attach tissue to medical devices such as heart valves, repair body organs, repair connective tissue, etc. Conventional surgical sutures may be made from known biocompatible materials, particularly synthetic and natural biocompatible polymeric materials, which may be non-absorbable or absorbable. Examples of synthetic non-absorbable polymeric materials useful to manufacture non-absorbable sutures include polyesters, polyolefins, polyvinylidene fluorides and polyamides. Examples of synthetic absorbable polymeric materials useful to manufacture absorbable sutures include polymers and copolymers made from lactones such as the lactides, glycolide, p-dioxanone, ε-caprolactone, and trimethylene carbonate. The term absorbable is meant to be a generic term, which may also include bioabsorbable, resorbable, bioresorbable, degradable or biodegradable.

Absorbable sutures are preferred by surgeons for use in many surgical procedures because of several advantages and properties possessed by such sutures. Absorbable sutures must be capable of providing the desired tensile strength in vivo for a sufficient period of time to allow for effective tissue healing. Wound healing is dependent on the nature of the specific tissue as well as the healing characteristics of the individual undergoing the surgical procedure. For example, poorly vascularized tissue is likely to heal more slowly than highly vascularized tissue; likewise, diabetic patients and the elderly tend to heal more slowly as well. There are thus opportunities to provide suture materials that can match the healing characteristics of a variety of wounds. Any implant, such as a suture, appears as a foreign body to the patient's immune system. Upon absorption of an absorbable suture the polymeric material comprising the suture is eliminated from the body, thus providing, it is believed, a better patient outcome. The outcome may be improved for several reasons including decreased post-operative pain, reduced risk of long-term infections, and better patient comfort. In addition, it is known that implantable medical devices, including sutures, may provide a platform for the attachment of bacteria and the subsequent formation of bacterial biofilms. The absorption and elimination of absorbable sutures may result in a significant diminishment of infections and decreased biofilm formation at the wound site.

Absorbable sutures are designed to have the requisite physical characteristics to assure desirable and efficacious in vivo behavior. Specifically, the sutures need to retain appropriate tensile strength during the required healing period; this is typically characterized as breaking strength retention (BSR). In order to obtain the required design properties, it is necessary to provide absorbable polymers and manufacturing processes that will yield absorbable sutures with the required properties.

Likewise, the retention of mechanical properties post-implantation is often a very important and critical feature of an absorbable medical device. The device must retain mechanical integrity until the tissue has healed sufficiently. In some bodily tissues, healing occurs more slowly, requiring an extended retention of mechanical integrity. As mentioned earlier, this is often associated with tissue that has poor vascularization. Likewise there are other situations in which a given patient may be prone to poor healing, e.g., the diabetic patient.

It is known to include certain medicants, such as antimicrobials, in sutures or other medical implants such as hernia meshes or the like, which must stay in vivo for extended periods of time, so as to reduce the likelihood of post-surgical infections. However, the presence of such substances incorporated into the polymer material of an implant, especially in particle form, can negatively affect its processibility and/or mechanical integrity.

Despite recent advances, there remains an unmet need in the art to provide medical devices with medicants while maintaining their mechanical strength and long term integrity.

### SUMMARY

Presented is an implantable medical device, comprising a collection of filaments, comprising a plurality of first variety filaments made of a first polymeric material; and at least one second variety filament, wherein the second variety filament is coated or impregnated with a biomedically useful agent.

In some forms, the medical device is a braided suture or a mesh.

In another form, the braided suture has mechanical properties within 10% of the mechanical properties of an equivalent braided suture having only the first variety of filaments, or even has mechanical properties substantially equivalent to the mechanical properties of an equivalent braided suture having only the first variety of filaments.

In yet another form, the biomedically useful agent comprises an antimicrobial agent, such as triclosan, chlorhexidine gluconate or glucose oxidase.

In still yet another form, either or both of the first variety filaments and the second variety filaments contains a pH modifying substance as the biomedically useful agent.

In a further form, the first and second polymeric materials are both absorbable and have different absorption profiles, such as wherein the second polymeric material absorbs faster than the first polymeric material, or wherein the second polymeric material absorbs slower than the first polymeric material.

Advantageously, the second variety filament has a high affinity to the biomedically useful agent, such as wherein the second variety filament comprises a polymeric material having a high solubility of biomedically useful agent.

In one form, the second variety filament is made of a second polymeric material.

In another form, the first and second polymeric materials comprise PLA, PGA, PCL, PLGA, PP, PE, PDS, or combinations or copolymers of the monomers thereof, and advantageously, the second polymeric material comprises at least 30 wt % polycaprolactone.

In another form, one of the first and second polymeric materials is absorbable and the other is non-absorbable.

In another form, the medical device can further comprise a coating surrounding the collection of filaments.

In some forms, the coating contains a chemical compound which interacts with the biomedically useful agent.

In other forms, the medical device can comprise at least two second-variety filaments, and the at least two second variety filaments are coated or impregnated with the same biomedically useful agent, or the at least two second variety filaments are coated or impregnated with different biomedically useful agents, which optionally have different release profiles.

Advantageously, the number of second variety filaments can be varied to vary a loading of the biomedically useful agent in the medical device.

In one form, one of the at least two second variety filaments is coated or impregnated with glucose and the other is coated or impregnated with glucose oxidase.

In another form, one of the at least two second variety filaments is coated or impregnated with an acidic agent and the other is coated or impregnated with an alkaline agent.

In another form, one of the at least two second variety filaments is coated or impregnated with a pH modifying agent which potentiates the biomedically useful agent.

Additionally presented is an implantable medical device, comprising a collection of filaments, including a plurality of first variety filaments made of a first polymeric material; and at least one second variety filament; and at least one third variety of filament, wherein the second variety filament is coated or impregnated with a first biomedically useful agent and the third variety of filament is coated or impregnated with a second biomedically useful agent different from the first biomedically useful agent.

In one form, the second variety filament is made of a second polymeric material, and the third variety filament is made of a third polymeric material.

In one form, the medical device is a braided suture or a mesh.

In another form, the first biomedically useful agent is an antimicrobial agent and the second biomedically useful agent is an antibiotic.

In another form, the first biomedically useful agent is a pH modifying agent and the second biomedically useful agent is an antibiotic.

In another form, the first biomedically useful agent is an antibiotic and the second biomedically useful agent is a different antibiotic, such as wherein the first biomedically useful agent is an antibiotic useful against gram positive bacteria and the second biomedically useful agent is an antibiotic useful against gram negative bacteria.

In some forms, at least the second and third polymeric materials are absorbable polymers, and can even have different absorption profiles. Likewise, the first biomedically useful agent can have a different release profile from that of the second biomedically useful agent.

Advantageously, the first biomedically useful agent is glucose and the second biomedically useful agent is glucose oxidase.

In another form, the first biomedically useful agent is an acidic agent and the second biomedically useful agent is an alkaline agent.

Alternatively, the first biomedically useful agent is a pH modifying agent and the second biomedically useful agent is an antimicrobial agent or an antibiotic agent.

Advantageously, the braided suture has mechanical properties within 10% of the mechanical properties of an equivalent braided suture having only the first variety of filaments, or even wherein the braided suture has mechanical properties substantially equivalent to the mechanical properties of an equivalent braided suture having only the first variety of filaments.

Additionally presented is a process for making an implantable medical device comprising a collection of filaments, comprising providing a plurality of first variety filaments made of a first polymeric material, providing at least one second variety filament, optionally providing at least one third variety of filament and combining the second variety filament and optional third variety filament with the plurality of first variety filaments, wherein the second variety filament, and if present the third variety filament is coated or impregnated with at least one biomedically useful agent.

In one form of the process, the second and optional third variety filaments are pre-coated or pre-impregnated with the biomedically useful agent(s) prior to combining them with the plurality of first variety filaments.

In one form, the second variety filament is made of a second polymeric material, and the optional third variety filament is made of an optional third polymeric material.

In another form, the process can further comprise applying a heat treatment and/or vacuum treatment to the medical device and redistributing the biomedically useful agent within the filaments, especially wherein the biomedically useful agent is triclosan and the heat treatment and/or vacuum treatment is part of a sterilization treatment.

In some forms, the biomedically useful agent(s) are compounded into the second and optional third polymeric materials prior to extruding the second variety filament and the optional third variety filament, such as wherein the biomedically useful agent comprises chlorhexidine gluconate.

Advantageously, the biomedically useful agent is glucose oxidase.

In one form, the second variety filament has a high affinity to the biomedically useful agent, and the process further comprises exposing the medical device to an environment containing the biomedically useful agent, such as wherein the second variety filament comprises at least about 30 wt % polycaprolactone and the biomedically useful agent is triclosan, and the triclosan is preferentially concentrated into the second variety filament. Accordingly, the process can further comprise applying a heat treatment and/or vacuum treatment to the medical device and redistributing the triclosan within the filaments.

In another form, the process can include pre-treating the second variety filament in a hot aqueous solution or by irradiation prior to coating or impregnating it with the biomedically useful agent.

Advantageously, the medical device made according to the process is a braided suture or a mesh.

Additionally presented is a method of implanting the medical device described above in a surgical procedure, such as tissue repair, tissue reinforcement or suturing.

### BRIEF DESCRIPTION OF THE DRAWINGS

The present disclosure is susceptible to various modifications and alternative forms, specific exemplary implementations thereof have been shown in the drawings and are herein described in detail. It should be understood, however, that the description herein of specific exemplary implementations is not intended to limit the disclosure to the particular forms disclosed herein.

This disclosure is to cover all modifications and equivalents as defined by the appended claims. It should also be understood that the drawings are not necessarily to scale, emphasis instead being placed upon clearly illustrating principles of exemplary embodiments of the present invention. Moreover, certain dimensions may be exaggerated to help visually convey such principles. Further where considered appropriate, reference numerals may be repeated among the drawings to indicate corresponding or analogous elements. Moreover, two or more blocks or elements depicted as distinct or separate in the drawings may be combined into a single functional block or element. Similarly, a single block or element illustrated in the drawings may be implemented as multiple steps or by multiple elements in cooperation.

The forms disclosed herein are illustrated by way of example, and not by way of limitation, in the figures of the accompanying drawings and in which like reference numerals refer to similar elements and in which:

FIG. 1 presents a schematic representation of a cross-section of a braided suture according to the present invention;

FIG. 2 presents a schematic representation of a cross-section of an alternative braided suture according to the present invention; and

FIG. 3 presents a schematic representation of a cross-section of another alternative braided suture according to the present invention.

### DETAILED DESCRIPTION

Various aspects will now be described with reference to specific forms selected for purposes of illustration. It will be appreciated that the spirit and scope of the apparatus, system and methods disclosed herein are not limited to the selected forms. Moreover, it is to be noted that the figures provided herein are not drawn to any particular proportion or scale, and that many variations can be made to the illustrated forms.

Each of the following terms written in singular grammatical form: “a,” “an,” and “the,” as used herein, may also refer to, and encompass, a plurality of the stated entity or object, unless otherwise specifically defined or stated herein, or, unless the context clearly dictates otherwise. For example, the phrases “a device,” “an assembly,” “a mechanism,” “a component,” and “an element,” as used herein, may also refer to, and encompass, a plurality of devices, a plurality of assemblies, a plurality of mechanisms, a plurality of components, and a plurality of elements, respectively.

Each of the following terms: “includes,” “including,” “has,” “'having,” “comprises,” and “comprising,” and, their linguistic or grammatical variants, derivatives, and/or conjugates, as used herein, means “including, but not limited to.”

Throughout the illustrative description, the examples, and the appended claims, a numerical value of a parameter, feature, object, or dimension, may be stated or described in terms of a numerical range format. It is to be fully understood that the stated numerical range format is provided for illustrating implementation of the forms disclosed herein, and is not to be understood or construed as inflexibly limiting the scope of the forms disclosed herein.

Moreover, for stating or describing a numerical range, the phrase “in a range of between about a first numerical value and about a second numerical value,” is considered equivalent to, and means the same as, the phrase “in a range of from about a first numerical value to about a second numerical value,” and, thus, the two equivalently meaning phrases may be used interchangeably.

It is to be understood that the various forms disclosed herein are not limited in their application to the details of the order or sequence, and number, of steps or procedures, and sub-steps or sub-procedures, of operation or implementation of forms of the method or to the details of type, composition, construction, arrangement, order and number of the system, system sub-units, devices, assemblies, sub-assemblies, mechanisms, structures, components, elements, and configurations, and, peripheral equipment, utilities, accessories, and materials of forms of the system, set forth in the following illustrative description, accompanying drawings, and examples, unless otherwise specifically stated herein. The apparatus, systems and methods disclosed herein can be practiced or implemented according to various other alternative forms and in various other alternative ways.

It is also to be understood that all technical and scientific words, terms, and/or phrases, used herein throughout the present disclosure have either the identical or similar meaning as commonly understood by one of ordinary skill in the art, unless otherwise specifically defined or stated herein. Phraseology, terminology, and, notation, employed herein throughout the present disclosure are for the purpose of description and should not be regarded as limiting.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, etc. and sub-ranges such as 10 to 50, 20 to 100, etc. Similarly, it should be understood that when numerical ranges are provided, such ranges are to be construed as providing literal support for claim limitations that only recite the lower value of the range as well as claims limitation that only recite the upper value of the range. For example, a disclosed numerical range of 10 to 100 provides literal support for a claim reciting “greater than 10” (with no upper bounds) and a claim reciting “less than 100” (with no lower bounds). In the figures, like numerals denote like, or similar, structures and/or features; and each of the illustrated structures and/or features may not be discussed in detail herein with reference to the figures. Similarly, each structure and/or feature may not be explicitly labeled in the figures; and any structure and/or feature that is discussed herein with reference to the figures may be utilized with any other structure and/or feature without departing from the scope of the present disclosure.

Braided structures are known to be composed of a plurality of individual filaments which are twisted into a cohesive bundle of the filaments, such as to form threads. Often, these bundles of filaments are further braided together to form yarns and/or knitted or woven into larger articles, such as meshes or fabrics.

The medical device industry utilizes the braiding process to form implantable medical devices, such as sutures and meshes. Frequently, the filaments which are used for medical devices are formed from biocompatible synthetic polymers which are divided into those which are biodegradable; those which “resorb” or “absorb” into the bodily tissues over time, and those which are biodurable; those which do not resorb or absorb, and retain their shape over time. The biodegradable polymers readily break down into small segments when exposed to moist body tissue. The segments then either are absorbed by the body, or passed by the body. More particularly, the biodegraded segments do not elicit permanent chronic foreign body reaction, because they are absorbed by the body or passed from the body, such that no permanent trace or residual of the segment is retained by the body.

Selection of the particular polymer or copolymer which is desirable for a medical device, such as a suture, depends on what type of tissue is being treated, and how long the healing process is expected to take, among other factors. A suture must have adequate BSR to be pulled through the particular tissue being approximated, and to maintain its mechanical integrity at least during the healing process.

However, as stated above, it is often desirable to incorporate certain medicants, such as antimicrobials, into or onto the suture material, which can be difficult to accurately load into the medical device/suture and/or negatively affect its processibility during manufacture or mechanical strength over time while in vivo.

The term “medicant” as used herein means any biomedically useful agent which has beneficial effects in vivo. The biomedically useful agents that may be incorporated in the surgical sutures of the present invention include antimicrobials, therapeutic agents, antibiotics, antiviral agents, anti-inflammatory agents, wound healing agents, beneficial cytokines, anti-cancer agents, analgesics and analgesic combinations, anorexics, antihelmintics, antiarthritics, antiasthmatic agents, adhesion preventatives, anticonvulsants, antidepressants, antidiuretic agents, antidiarrheals, antihistamines, anti-inflammatory agents, antimigraine preparations, contraceptives, antinauseants, antineoplastics, antiparkinsonism drugs, antipruritics, antipsychotics, antipyretics, antispasmodics, anticholinergics, sympathomimetics, xanthine derivatives, and pH modifiers.

If desired, the sutures or webs of the present invention may contain other conventional medically useful components and agents. The other components, additives or agents will be present to provide additional desired characteristics to the sutures of the present invention including but not limited to controlled drug elution, therapeutic aspects, radio-opacification, and enhanced osseointegration.

The surgical sutures or webs can also include other conventional additives including dyes, radio-opaque agents, growth factors and the like. The dye should be generally acceptable for clinical use with absorbable polymers; this includes, without limitation, D&C Violet No. 2 and D&C Blue No. 6 and similar combinations thereof. Additional dyes that are useful include conventional dyes useful with absorbable polymers including D&C Green No. 6, and D&C Blue No. 6.

Typically, the amount of the other adjuncts will be about 0.1 weight percent to about 20 weight percent, more typically about 1 weight percent to about 10 weight percent and preferably about 2 weight percent to about 5 weight percent.

In order to address these competing factors, it has been found that medical devices made from filament bundles, such as braided sutures or woven or knitted meshes, can be formed using at least two different filamentary materials, at least a first variety of filaments in a major portion, and a second variety of filaments in a minor portion incorporating a biomedically useful agent, such that the presence of the minor portion of filaments, which can have less mechanical strength than those of the major portion, do not drastically diminish the overall mechanical strength or BSR of the medical device.

The present invention is directed to an implantable medical device, comprising a collection of filaments, including a plurality of first variety filaments made of a first polymeric material, and at least one second variety filament, which can optionally be made of a second polymeric material, wherein the second variety filament is coated or impregnated with a biomedically useful agent. In some forms, the medical device is a braided suture or a mesh.

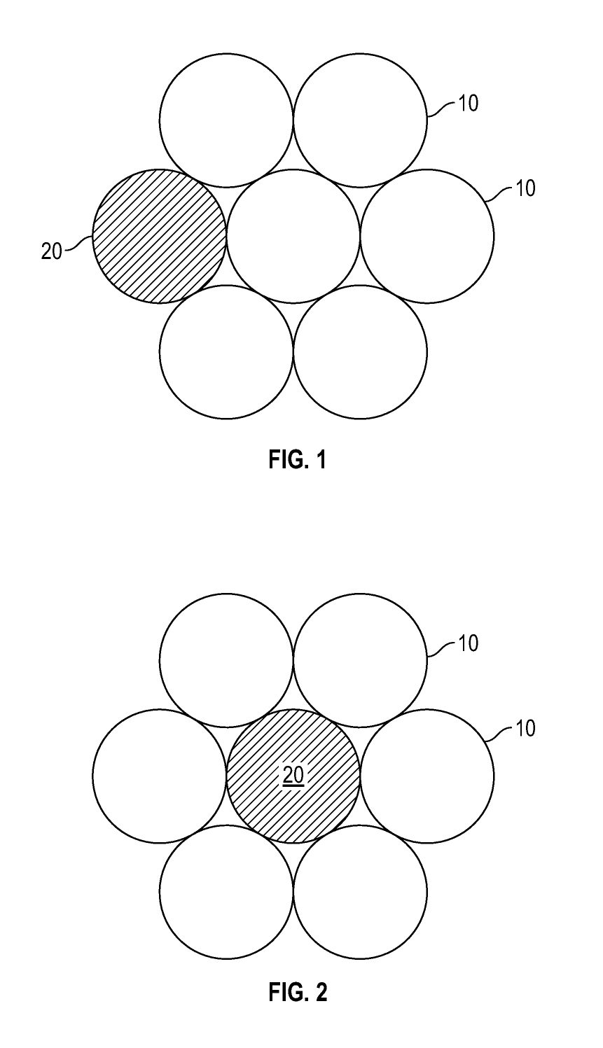

FIG. 1 presents a simplified schematic representation of a cross-section of a braided suture according to the present invention, which is a collection of filaments having a plurality of first variety filaments 10 and a single second variety filament 20, which is understood to include the biomedically useful agent. In this case, the second variety filament is located on the outside of the bundle.

FIG. 2 presents a simplified schematic representation of a cross-section of an alternative braided suture according to the present invention, wherein the second variety filament 20 is located in the center of the bundle and is surrounded by the plurality of first variety filaments 10. Of course, FIGS. 1 and 2 are simplified representations, as braided sutures usually have many more filaments than are depicted in the figures.

It has been found that a braided suture of the structure described above has mechanical properties within 10% of the mechanical properties of an equivalent braided suture having only the first variety of filaments, or even has mechanical properties substantially equivalent to the mechanical properties of an equivalent braided suture having only the first variety of filaments.

In one form, the polymers used for making the medical devices herein are absorbable polymers. The term absorbable is meant to be a generic term, which may also include bioabsorbable, resorbable, bioresorbable, degradable or biodegradable.

In one form, the first and second variety filaments are made of first and second polymeric materials which are both absorbable and have different absorption profiles, such as wherein the second polymeric material absorbs faster than the first polymeric material, or wherein the second polymeric material absorbs slower than the first polymeric material.

Suitable biocompatible, biodegradable polymers may be synthetic or natural polymers. Suitable synthetic biocompatible, biodegradable polymers include polymers selected from the group consisting of aliphatic polyesters, poly(amino acids), copoly(ether-esters), polyalkylene oxalates, tyrosine-derived polycarbonates, poly(iminocarbonates), polyorthoesters, polyoxaesters, polyamidoesters, polyoxaesters containing amine groups, poly(anhydrides), polyphosphazenes, and combinations thereof.

For the purposes of this invention aliphatic polyesters include, but are not limited to, homopolymers and copolymers of lactide (which includes lactic acid, D-, L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, and blends thereof.

Suitable natural polymers include, but are not limited to collagen, elastin, hyaluronic acid, laminin, and gelatin, keratin, chondroitin sulfate and decellularized tissue.

Suitable bioabsorbable, biocompatible elastomeric copolymers include but are not limited to copolymers of ε-caprolactone and glycolide (preferably having a mole ratio of ε-caprolactone to glycolide of from about 30:70 to about 70:30, preferably 35:65 to about 65:35, and more preferably 45:55 to 35:65); elastomeric copolymers of ε-caprolactone and lactide, including L-lactide, D-lactide blends thereof or lactic acid copolymers (preferably having a mole ratio of ε-caprolactone to lactide of from about 35:65 to about 65:35 and more preferably 45:55 to 30:70) elastomeric copolymers of p-dioxanone (1,4-dioxan-2-one) and lactide including L-lactide, D-lactide and lactic acid (preferably having a mole ratio of p-dioxanone to lactide of from about 40:60 to about 60:40); elastomeric copolymers of ε-caprolactone and p-dioxanone (preferably having a mole ratio of epsilon-caprolactone to p-dioxanone of from about 30:70 to about 70:30); elastomeric copolymers of p-dioxanone and trimethylene carbonate (preferably having a mole ratio of p-dioxanone to trimethylene carbonate of from about 30:70 to about 70:30); copolymers of trimethylene carbonate and glycolide (preferably having a mole ratio of trimethylene carbonate to glycolide of from about 30:70 to about 70:30); elastomeric copolymer of trimethylene carbonate and lactide including L-lactide, D-lactide, blends thereof or lactic acid copolymers (preferably having a mole ratio of trimethylene carbonate to lactide of from about 30:70 to about 70:30) and blends thereof. In one embodiment, the elastomeric copolymer is a copolymer of glycolide and ε-caprolactone. In another embodiment, the elastomeric copolymer is a copolymer of lactide and ε-caprolactone.

In another form, one of the first and second polymeric materials is absorbable and the other is non-absorbable; i.e. biodurable. For example, the first polymeric material can be polyethylene, polypropylene or copolymers of the monomers thereof, and the second polymeric material can be one of the biodegradable polymers mentioned above.

Suitable biodurable polymers include, but are not limited to polyurethane, polypropylene (PP), polyethylene (PE), polycarbonate, polyamides, such as nylon, polyvinylchloride (PVC), polymethyl-metacrylate (PMMA), polystyrene (PS), polyester, polyetheretherketone (PEEK), polytetrafluoroethylene (PTFE), polytrifluorochloroethylene (PTFCE), polyvinylfluoride (PVF), fluorinated ethylene propylene (FEP), polyacetal, polysulf one, silicons, and combinations thereof.

Advantageously, the first and second polymers are selected from poly(lactic acid) (PLA), polyglycolic acid (PGA), polycaprolactone (PCL), polylactide-glycolic acid (PLGA), polypropylene (PP), polyethylene (PE), polydioxanone (PDS), or combinations or copolymers of the monomers thereof, and advantageously, the second polymeric material comprises at least 30 wt % polycaprolactone.

One advantage of including a medicant on absorbable second polymer filaments is that the medicant can be delivered throughout the length or breadth of the medical device, and depending upon the absorption profile of the second polymer, the medicant can be delivered at either a fast or slow rate.

Another advantage is that the number of second variety filaments can be varied to vary a loading of the biomedically useful agent in the medical device. In this way, the dosage of the biomedically useful agent can be increased by adding more of the medicant-loaded filaments into the filament bundle.

Additionally, the position of the second variety filament having the medicant, either on the inside (FIG. 2) or the outside (FIG. 1) of the filament bundle, can increase or decrease the rate that the medicant is delivered to the surrounding tissue.

Suitable antimicrobial agents may be selected from, but are not limited to, halogenated hydroxyl ethers, acyloxydiphenyl ethers, or combinations thereof. In particular, the antimicrobial agent may be a halogenated 2-hydroxy diphenyl ether and/or a halogenated 2-acyloxy diphenyl ether, esters of acetic acid, chloroacetic acid, methyl or dimethyl carbamic acid, benzoic acid, chlorobenzoic acid, methylsulfonic acid and chloromethylsulfonic acid are particularly suitable. Some particularly advantageous antimicrobial agents are 2,4,4′-trichloro-2′-hydroxydiphenyl ether, commonly referred to as triclosan, chlorhexidine gluconate and glucose oxidase.

In addition to the antimicrobial agents described above, the second variety filaments of the medical device may have a biocide, a disinfectant and/or an antiseptic, including but not limited to alcohols such as ethanol and isopropanol; aldehydes such as glutaraldehyde and formaldehyde; anilides such as triclorocarbanilide; biguanides such as chlorhexidine; chlorine-releasing agents such as sodium hypochlorite, chlorine dioxide and acidified sodium chlorite; iodine-releasing agents such as povidone-iodine and poloxamer-iodine; metals such as silver nitrate, silver sulfadiazine, other silver agents, copper-8-quinolate and bismuth thiols; peroxygen compounds such as hydrogen peroxide and peracetic acid; phenols; quaternary ammonium compounds such as benzalkonium chloride, cetrimide and ionenes-polyquaternary ammonium compounds.

The second variety filaments of the medical device may have antibiotics, including but not limited to penicillins such as amoxicillin, oxacillin and piperacillin; cephalosporins parenteral such as cefazolin, cefadroxil, cefoxitin, cefprozil, cefotaxime and cefdinir; monobactams such as aztreonam; beta-lactamase inhibitors such as clavulanic acid sulbactam; glycopeptide such as vancomycin; polymixin; quinolones such as nalidixic acid, ciprofloxacin and levaquin; metranidazole; novobiocin; actinomycin; rifampin; aminoglycosides such as neomycin and gentamicin; tetracyclines such as doxycycline; chloramphenicol; macrolide such as erythromycin; clindamycin; sulfonamide such as sulfadiazine; trimethoprim; topical antibiotics; bacitracin; gramicidin; mupirocin; and/or fusidic acid.

The second variety filaments of the medical device may have antimicrobial peptides such as defensins, magainin and nisin; lytic bacteriophage; surfactants; adhesion blockers such as antibodies, oligosaccharides and glycolipids; oligonucleotides such as antisense RNA; efflux pump inhibitors; photosensitive dyes such as porphyrins; immune modulators such as growth factors, interleukins, interferons and synthetic antigens; and/or chelators such as EDTA, sodium hexametaphosphate, lactoferrin and transferrin.

Advantageously, the second variety filament has a high affinity to the biomedically useful agent, such as wherein the second variety filament comprises a material having a high solubility of biomedically useful agent, for example where the second variety filament comprises at least about 30 wt % polycaprolactone and the biomedically useful agent is triclosan. Triclosan is known to vaporize under relatively mild temperature and/or low pressure conditions, and will be preferentially absorbed into the polycaprolactone-containing polymer.

In still yet another form, either or both of the first variety filaments and the second variety filaments contains a pH modifying substance as the biomedically useful agent. For example, the combination of a pH modifier, such as NaCO₃, with an antibiotic can be synergistic, since some anti-bacterial agents are potentiated at higher pH by stressing bacteria, resulting in higher susceptibility to the antibacterial agent. Also, alkaline agents can also help to neutralize the acidic hydrolysis products of most absorbable polymers, like PLGA, and help accelerate absorption.

Likewise, it is known that oxidized regenerated cellulose (ORC), which is acidic, demonstrates antibacterial activity and suggests that lowering the pH at the location of the medical device could likewise stress bacteria and potentiate anti-microbial agents.

Suitable pH modification can be provided by the presence of polymeric monomers, endcapping polymers by acidic or basic groups, weakening of the polymer by hydrotreatment or by irradiation which will result in faster hydrolysis and thus will generate faster acidification, and by other similar techniques. Admixing of short polymeric chains into the base polymer, with the short chains endcapped by alkaline or acidic groups can be utilized. Other useful acidic compounds include boric acid, benzoic acid, uric acid and urate salts, lactic acid, including D-lactic acid and L-lactic acid, and other known acidic compounds, preferably solid or semi-solid.

Additionally, pH modification can be achieved by using any number of known basic or alkaline agents added or compounded into the polymers, including hydroxides, salts of strong base and weak acid or salts of strong acid and weak base, and combinations thereof. Carbonates, TRIS, borates, glycine, phosphates, methylamine, 2-(Cyclohexylamino)ethanesulfonic acid (CHES), 3-(Cyclohexylamino)-1-propanesulfonic acid (CAPS) or 3-(Cyclohexylamino)-2-hydroxy-1-propanesulfonic acid (CAPSO) can be used. Useful alkaline components include: NaOH sodium hydroxide, Na₂CO₃ sodium carbonate, NaHCO₃ sodium bicarbonate, Na₃BO₃, sodium borate, Na₃CH₃COO, sodium acetate, Na₃PO₄, Na₂HPO₄, NaH₂PO₄ sodium phosphates, and similar. Salts of potassium or any other metal can also be used instead of sodium. Ammonia cation NH4⁺ can also be used in the salts. Also useful are combinations of salt and corresponding hydroxide.

Alternatively, the medical device can further include a coating surrounding the collection of filaments, such that the biomedically useful agent is separated from the coating. Such a structure can be advantageous when the biomedically useful agent is unstable in the presence of the coating material, or vice versa. In this way, interaction between the components of the biomedically useful agent and the coating can be delayed until the medical device is in place in the patient. The coating composition can include any of the antimicrobial agents, antibiotic agents or a pH modifying agent, as listed above. Advantageously, the coating contains a chemical compound which favorably interacts with the biomedically useful agent, such as those potentiating agents mentioned above.

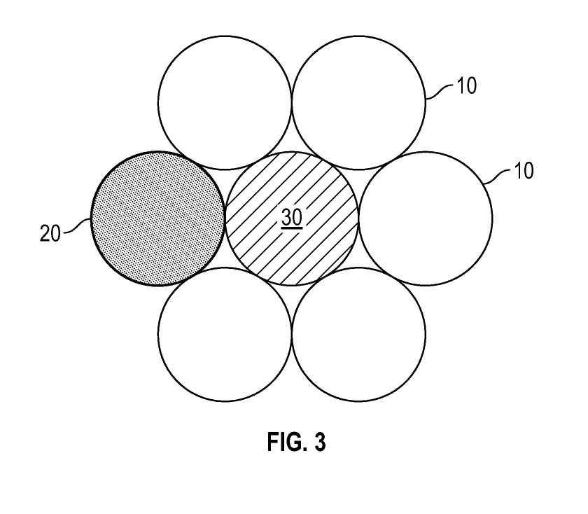

In another form, the medical device can further comprise a third variety of filament, optionally made of a third polymeric material, wherein the third variety filament is coated or impregnated with a second biomedically useful agent different from the first biomedically useful agent coated or impregnated on the second variety filament. FIG. 3 illustrates a non-limiting example of this embodiment, wherein the plurality of first variety filaments 10 has included therewith at least one second variety filament 20 and at least one third variety filament 30. The physical arrangement of filaments 20 and 30 can be varied, as can their numbers within the structure. It should be understood that while the third variety of filament can differ from the second variety of filament by being different polymers, both can be made of the same polymer and differ by the nature of the biolomedically useful agent coated on or impregnated in the filaments.

Again, such a structure can be advantageous when the first biomedically useful agent is unstable in the presence of the second biomedically useful agent, and interaction between the components of the different biomedically useful agents can be delayed until the medical device is in place in the patient and release of the biomedically useful agents begins. The first and second biomedically useful agents can include any of the antimicrobial agents, antibiotic agents, pH modifying agents or potentiating agents, as listed above, and the second and third polymeric materials can both be different polymers, such as wherein both are absorbable polymers which have different absorption profiles. Suitable polymers can be selected from those listed above. Likewise, the first and second biomedically useful agents can be selected to have different release profiles.

In one particularly advantageous form, the first biomedically useful agent is glucose and the second biomedically useful agent is glucose oxidase, which when combined form hydrogen peroxide in vivo.

In another form, first biomedically useful agent is an acidic agent and the second biomedically useful agent is an alkaline agent, which when combined can be used to maintain a consistent pH at the location of the medical device. Both the acidic and alkaline agents can accelerate resorption of the absorbable polymer filaments as catalysts for hydrolysis, but having excess acid or base could result in an adverse tissue reaction. So having these mutually neutralizing materials in proximity will accelerate suture hydrolysis at the micro-level, while maintaining overall bulk pH neutrality.

In another advantageous form, the first biomedically useful agent is an antibiotic and the second biomedically useful agent is a different antibiotic, such as wherein the first biomedically useful agent is an antibiotic useful against gram positive bacteria and the second biomedically useful agent is an antibiotic useful against gram negative bacteria.

Again, since there are relatively so few second and third variety filaments among the filament bundle(s), the medical device, such as a braided suture, has mechanical properties within 10% of the mechanical properties of an equivalent braided suture having only the first variety of filaments, or even wherein the braided suture has mechanical properties substantially equivalent to the mechanical properties of an equivalent braided suture having only the first variety of filaments.

Another form of the invention is directed to a process for making an implantable medical device comprising a collection of filaments, comprising providing a plurality of first variety filaments made of a first polymeric material, providing at least one second variety filament, which can be made of a second polymeric material, and combining the second variety filament with the plurality of first variety filaments, wherein said second variety filament is coated or impregnated with a biomedically useful agent. The combining step can be conducted by winding or twisting the separate filaments together into the form of a thread, or winding or twisting a collection of such threads together to form a braided suture, and optionally knitting or weaving the threads into a two-dimensional or areal form, such as a mesh. It should be understood that, as described above, the collection of filaments can include at least one third variety filament, which can be made of a third polymeric material which is coated or impregnated with a second biomedically useful agent.

The process can include a step wherein the second variety filament is pre-coated or pre-impregnated with said biomedically useful agent prior to combining it with the plurality of first variety filaments. Additionally, when the biomedically useful agent is relatively volatile, such as halogenated hydroxyl ethers, acyloxydiphenyl ethers, the former including triclosan, it can be advantageous to apply a heat treatment and/or vacuum treatment to the medical device to redistribute the biomedically useful agent within the filaments. Conveniently, this heat treatment and/or vacuum treatment can be part of a sterilization treatment, such as ethylene oxide sterilization.

In some forms, the biomedically useful agent can be compounded into the second polymeric material prior to extruding the second variety filament, such as where the biomedically useful agent comprises chlorhexidine gluconate, or glucose oxidase.

In another form, when the second variety filament has a high affinity to said biomedically useful agent, the process can further comprise exposing the medical device to an environment containing said biomedically useful agent, such as wherein the second variety filament comprises at least about 30 wt % polycaprolactone and the biomedically useful agent is triclosan. In this form, the triclosan is preferentially concentrated into the second variety filament. If desired, the process can further comprise applying a heat treatment and/or vacuum treatment to the medical device and redistributing the triclosan within the filaments.

Alternatively, the process can include pre-treating the second variety filament in a hot aqueous solution or by irradiation prior to coating or impregnating it with said biomedically useful agent.

### EXAMPLES

Further illustrative, non-exclusive examples of systems and methods according to the present disclosure are presented in the following enumerated paragraphs. It is within the scope of the present disclosure that an individual step of a method recited herein, including in the following enumerated paragraphs, may additionally or alternatively be referred to as a “step for” performing the recited action.

PCT1. An implantable medical device, comprising a collection of filaments, comprising a plurality of first variety filaments made of a first polymeric material; and at least one second variety filament, wherein said second variety filament is coated or impregnated with a biomedically useful agent.

PCT2. The medical device of paragraph PCT1, wherein the medical device is a braided suture or a mesh.

PCT3. The medical device of paragraph PCT1 or PCT2, which is a braided suture which has mechanical properties within 10% of, or substantially equal to the mechanical properties of an equivalent braided suture having only the first variety of filaments.

PCT4. The medical device of any of paragraphs PCT1 to PCT3, wherein said biomedically useful agent comprises an antimicrobial agent, such as triclosan, chlorhexidine gluconate or glucose oxidase.

PCT5. The medical device of any of paragraphs PCT1 to PCT4, wherein either or both of the first variety filaments and the second variety filaments contains a pH modifying agent.

PCT6. The medical device of any of paragraphs PCT1 to PCT5, wherein the second variety filament is made of a second polymeric material.

PCT7. The medical device of any of paragraphs PCT1 to PCT6, wherein the first and second polymeric materials comprise PLA, PGA, PCL, PLGA, PP, PE, PDS, or combinations or copolymers of monomers thereof.

PCT8. The medical device of any of paragraphs PCT1 to PCT7, wherein one of the first and second polymeric materials is absorbable and the other is non-absorbable, or wherein the first and second polymeric materials are both absorbable and have different absorption profiles.

PCT9. The medical device of any of paragraphs PCT1 to PCT8, further comprising at least one third variety of filament optionally made of an optional third polymeric material, wherein said second variety filament is coated or impregnated with a first biomedically useful agent and said third variety of filament is coated or impregnated with a second biomedically useful agent different from the first biomedically useful agent.

PCT10. The medical device of paragraph PCT9, wherein the first biomedically useful agent is an antimicrobial agent and the second biomedically useful agent is an antibiotic, or wherein the first biomedically useful agent is a pH modifying agent and the second biomedically useful agent is an antibiotic, or wherein the first biomedically useful agent is an antibiotic and the second biomedically useful agent is a different antibiotic.

PCT11. The medical device of paragraph PCT9 or PCT10, wherein at least the second and optional third polymeric materials are absorbable polymers and have different absorption profiles, and optionally wherein the first biomedically useful agent has a different release profile from that of the second biomedically useful agent.

PCT12. The medical device of any of paragraphs PCT9 to PCT11, wherein the first biomedically useful agent is glucose and the second biomedically useful agent is glucose oxidase, or wherein the first biomedically useful agent is an acidic agent and the second biomedically useful agent is an alkaline agent, or wherein the first biomedically useful agent is a pH modifying agent and the second biomedically useful agent is an antimicrobial agent or an antibiotic agent.

PCT13. A process for making an implantable medical device comprising a collection of filaments, comprising providing a plurality of first variety filaments made of a first polymeric material, providing at least one second variety filament, optionally providing at least one third variety of filament and combining the second variety filament and optional third variety filament with the plurality of first variety filaments, wherein the second variety filament, and if present the third variety filament is coated or impregnated with at least one biomedically useful agent.

PCT14. The process of paragraph PCT13, wherein the second variety filament is made of a second polymeric material, and the optional third variety filament is made of a third polymeric material.

PCT15. The process of paragraph PCT13 or PCT14, wherein the second variety filament and optional third variety filament are pre-coated or pre-impregnated with said biomedically useful agent(s) prior to combining them with the plurality of first variety filaments.

PCT16. The process of any of paragraphs PCT13 to PCT15, further comprising applying a heat treatment and/or vacuum treatment to the medical device and redistributing the biomedically useful agent within the filaments.

PCT17. The process of any of paragraphs PCT13 to PCT16, wherein the biomedically useful agent is triclosan.

PCT18. The process of any of paragraphs PCT11 to PCT17, wherein the heat treatment and/or vacuum treatment is part of a sterilization treatment.

PCT19. The process of any of paragraphs PCT11 to PCT18, wherein said biomedically useful agent(s) are compounded into the second and optional third polymeric materials prior to extruding the second variety filament and the optional third variety filament.

PCT20. The process of any of paragraphs PCT11 to PCT18, wherein the second variety filament has a high affinity to said biomedically useful agent, and further comprising exposing the medical device to an environment containing said biomedically useful agent, so as to concentrate the biomedically useful agent into the second variety filament having a high affinity to the biomedically useful agent.

PCT21. The process of any of paragraphs PCT11 to PCT20, further comprising pre-treating the second variety filament in a hot aqueous solution or by irradiation prior to coating or impregnating it with the biomedically useful agent.

PCT22. The process of any of paragraphs PCT11 to PCT21, wherein the at least one biomedically useful agent is selected from triclosan, chlorhexidine gluconate or glucose oxidase.

PCT23. A method of implanting the medical device described in any of paragraphs PCT1 to PCT12 in a surgical procedure, such as tissue repair, tissue reinforcement or suturing.

### INDUSTRIAL APPLICABILITY

The systems and methods disclosed herein are applicable to the medical device industry.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

What is claimed:

1. An implantable medical device, comprising:
a collection of filaments, comprising:
a plurality of first variety filaments made of a first polymeric material; and
at least one second variety filament different from the first variety filaments,
wherein said second variety filament is coated or impregnated with a biomedically useful agent.

2. The medical device of claim 1, which is a braided suture.

3. The medical device of claim 2, wherein the braided suture has mechanical properties within 10% of the mechanical properties of an equivalent braided suture having only the first variety of filaments.

4. The medical device of claim 2, wherein the braided suture has mechanical properties substantially equivalent to the mechanical properties of an equivalent braided suture having only the first variety of filaments.

5. The medical device of claim 1, which is a mesh.

6. The medical device of claim 1, wherein said biomedically useful agent comprises an antimicrobial agent.

7. The medical device of claim 6, wherein said antimicrobial agent comprises triclosan.

8. The medical device of claim 6, wherein said antimicrobial agent comprises chlorhexidine gluconate.

9. The medical device of claim 1, wherein said biomedically useful agent comprises glucose oxidase.

10. The medical device of claim 1, wherein either or both of the first variety filaments and the second variety filaments contains a pH modifying agent.

11. The medical device of claim 1, wherein the second variety of filaments is made of a second polymeric material.

12. The medical device of claim 11, wherein the first and second polymeric materials are both absorbable and have different absorption profiles.

13. The medical device of claim 12, wherein the second polymeric material absorbs faster than the first polymeric material.

14. The medical device of claim 12, wherein the second polymeric material absorbs slower than the first polymeric material.

15. The medical device of claim 12, wherein the second variety filament has a high affinity to said biomedically useful agent.

16. The medical device of claim 12, wherein the second variety filament comprises a polymeric material having a high solubility of biomedically useful agent.

17. The medical device of claim 12, wherein the second polymeric material comprises at least 30 wt % polycaprolactone.

18. The medical device of claim 12, wherein the first and second polymeric materials comprise PLA, PGA, PCL, PLGA, PP, PE, PDS or combinations or copolymers of monomers thereof.

19. The medical device of claim 12, wherein one of the first and second polymeric materials is absorbable and the other is non-absorbable.

20. The medical device of claim 1, further comprising a coating surrounding said collection of filaments.

21. The medical device of claim 20, wherein said coating contains a chemical compound which interacts with said biomedically useful agent.

22. The medical device of claim 1, which comprises at least two second-variety filaments.

23. The medical device of claim 22, wherein the at least two second variety filaments are coated or impregnated with the same biomedically useful agent.

24. The medical device of claim 23, wherein a number of second variety filaments is varied to vary a loading of the biomedically useful agent in the medical device.

25. The medical device of claim 22, wherein the at least two second variety filaments are coated or impregnated with different biomedically useful agents, which optionally have different release profiles.

26. The medical device of claim 22, wherein one of the at least two second variety filaments is coated or impregnated with glucose and the other is coated or impregnated with glucose oxidase.

27. The medical device of claim 22, wherein one of said at least two second variety filaments is coated or impregnated with an acidic agent and the other is coated or impregnated with an alkaline agent.

28. The medical device of claim 22, wherein one of said at least two second variety filaments is coated or impregnated with a pH modifying agent which potentiates the biomedically useful agent.

29. An implantable medical device, comprising:
a collection of filaments, comprising:
a plurality of first variety filaments made of a first polymeric material; and
at least one second variety filament; and
at least one third variety of filament,
wherein said second variety filament is coated or impregnated with a first biomedically useful agent and said third variety of filament is coated or impregnated with a second biomedically useful agent different from the first biomedically useful agent.

30. The medical device of claim 29, wherein the second variety filament is made of a second polymeric material, and the third variety filament is made of a third polymeric material.

31. The medical device of claim 29, which is a braided suture or a mesh.

32. The medical device of claim 29, wherein the first biomedically useful agent is an antimicrobial agent and the second biomedically useful agent is an antibiotic.

33. The medical device of claim 29, wherein the first biomedically useful agent is a pH modifying agent and the second biomedically useful agent is an antibiotic.

34. The medical device of claim 29, wherein the first biomedically useful agent is an antibiotic and the second biomedically useful agent is a different antibiotic.

35. The medical device of claim 32, wherein the first biomedically useful agent is an antibiotic useful against gram positive bacteria and the second biomedically useful agent is an antibiotic useful against gram negative bacteria.

36. The medical device of claim 29, wherein at least the second and third polymeric materials are absorbable polymers.

37. The medical device of claim 29, wherein at least the second and third polymeric materials are absorbable polymers and have different absorption profiles.

38. The medical device of claim 29, wherein the first biomedically useful agent has a different release profile from that of the second biomedically useful agent.

39. The medical device of claim 29, wherein the first biomedically useful agent is glucose and the second biomedically useful agent is glucose oxidase.

40. The medical device of claim 29, wherein the first biomedically useful agent is an acidic agent and the second biomedically useful agent is an alkaline agent.

41. The medical device of claim 29, wherein the first biomedically useful agent is a pH modifying agent and the second biomedically useful agent is an antimicrobial agent or an antibiotic agent.

42. The medical device of claim 31, wherein the braided suture has mechanical properties within 10% of the mechanical properties of an equivalent braided suture having only the first variety of filaments.

43. The medical device of claim 31, wherein the braided suture has mechanical properties substantially equivalent to the mechanical properties of an equivalent braided suture having only the first variety of filaments.

44. A process for making an implantable medical device comprising a collection of filaments, comprising:
providing a plurality of first variety filaments made of a first polymeric material;
providing at least one second variety filament;
optionally providing at least one third variety of filament; and
combining the second variety filament and optional third variety filament with the plurality of first variety filaments,
wherein said second variety filament, and if present said third variety filament is coated or impregnated with at least one biomedically useful agent.

45. The process of claim 44, wherein the second variety and optional third variety filaments are pre-coated or pre-impregnated with said biomedically useful agent(s) prior to combining them with the plurality of first variety filaments.

46. The process of claim 45, further comprising applying a heat treatment and/or vacuum treatment to the medical device and redistributing the biomedically useful agent within the filaments.

47. The process of claim 46, wherein the biomedically useful agent is triclosan.

48. The process of claim 47, wherein the heat treatment and/or vacuum treatment is part of a sterilization treatment.

49. The process of claim 43, wherein the second variety filament is made of a second polymeric material, and the optional third variety filament is made of a third polymeric material.

50. The process of claim 49, wherein said biomedically useful agent(s) are compounded into the second and optional third polymeric materials prior to extruding the second variety filament and the optional third variety filament.

51. The process of claim 44, wherein said biomedically useful agent comprises chlorhexidine gluconate.

52. The process of claim 44, wherein said biomedically useful agent is glucose oxidase.

53. The process of claim 44, wherein the second variety filament has a high affinity to said biomedically useful agent, and further comprising exposing the medical device to an environment containing said biomedically useful agent.

54. The process of claim 53, wherein the second variety filament comprises at least about 30 wt % polycaprolactone and the biomedically useful agent is triclosan, and said triclosan is preferentially concentrated into said second variety filament.

55. The process of claim 53, further comprising applying a heat treatment and/or vacuum treatment to the medical device and redistributing the triclosan within the filaments.

56. The process of claim 53, further comprising pre-treating said second variety filament in a hot aqueous solution or by irradiation prior to coating or impregnating it with said biomedically useful agent.

57. The process of claim 44, wherein said medical device is a suture.

58. The process of claim 44, wherein said medical device is a mesh.

59. The process of claim 44, wherein said medical device is a braided or twisted suture.

60. A method of implanting a medical device according to claim 1 in a surgical procedure.

61. The method of claim 60, wherein the surgical procedure is tissue repair.

62. The method of claim 60, wherein the surgical procedure is tissue reinforcement.

63. The method of claim 60, wherein the surgical procedure is suturing.
